(19) United States
(12) Patent Application Publication
(10) Pub. No.: US 2023/0030074 A1
(43) Pub. Date: Feb. 2, 2023

(54) TECHNIQUES FOR MAGNETOCALORIC THERAPY

(71) Applicant: Friscket Holdings LLC, Winter Park, FL (US)

(72) Inventor: James Baumgartner, Winter Park, FL (US)

(73) Assignee: Friscket Holdings LLC, Winter Park, FL (US)

(21) Appl. No.: 17/813,471

(22) Filed: Jul. 19, 2022

Related U.S. Application Data
(60) Provisional application No. 63/226,996, filed on Jul. 29, 2021.

(51) Int. Cl. A61N 2/00

(52) U.S. Cl. CPC A61N 2/004; A61N 2/002

(57) ABSTRACT

The disclosure relates to techniques for treating infiltrative tumors using magnetocaloric substances. After tumor removal, a vector carrying a magnetocaloric substance is introduced into a target area in a patient's body. After a period of time, the vectors migrate to cancerous cells and surround the tumor with the magnetocaloric substance. The patient is then exposed to a pulsing magnetic field that is controlled to heat the magnetocaloric substance to a target temperature. The temperature is maintained for a target duration that is sufficient to kill the cancerous cells.

### CROSS-REFERENCES TO RELATED APPLICATIONS

This application claims the benefit of provisional application 63/226,996, filed Jul. 29, 2021. This application is hereby incorporated by reference in its entirety for all purposes.

### BACKGROUND OF THE INVENTION

The present invention relates generally to the field of treatments for infiltrative tumors.

### BRIEF SUMMARY OF THE INVENTION

Certain embodiments of the present invention can provide methods, systems, and apparatuses for treating infiltrative tumors (e.g., glioblastoma) with magnetocaloric therapy. Other application embodiments are also described herein.

Some embodiments relate to a method for treating infiltrative tumors. The method includes administering a magnetocaloric vector to a patient in response to a determination of the presence of an infiltrative tumor. Administering the magnetocaloric treatment includes preparing an magnetocaloric vector. The dose of magnetocaloric vector is delivered to a target location. After waiting for the magnetocaloric vector to migrate to the infiltrative tumor cells, the infiltrative brain tumor cells are heated via the application of a pulsed magnetic field to the patient's tissue surrounding the target location.

Other embodiments relate to variations of a method for killing infiltrative brain tumors. The method further includes generating a magnetic field around the patient's tissue surrounding the target location. A set of vector clusters are identified and the temperature at the set of vector clusters are measured. The magnetic field is stopped after a target temperature at the set of vector clusters has been reached for a target duration.

Additional embodiments include, when the magnetocaloric vector contains a magnetocaloric metal, observing a first metal distribution using imaging. Until a difference between the first metal distribution and a second metal distribution is below a threshold, administering a second magnetocaloric treatment. After administering the second treatment, the second metal distribution is observed using imaging, and the difference between the first metal distribution and the second metal distribution is compared to a threshold.

Some embodiments include preparing the magnetocaloric vector includes obtaining an autologous stem cell sample from the patient. The autologous stem cell sample is processed to isolate the autologous stem cells and the stem cells are induced to phagocytize a magnetocaloric metal.

Additional embodiments can further include identifying an insertion point on the skull. An incision is created at the insertion point and the scalp is retracted. Once the scalp is retracted, an opening is created in the skull at the insertion point.

Some embodiments further include removing a tissue sample from a patient. The tissue sample is analyzed using standard histopathologic and detailed genetic analysis to determine if the tissue sample contains cells from an infiltrative tumor.

Reference to the remaining portions of the specification, including the drawings and claims, will realize other features and advantages of embodiments of the present invention. Further features and advantages, as well as the structure and operation of various embodiments of the present invention, are described in detail below with respect to the accompanying drawings. In the drawings, like reference numbers can indicate identical or functionally similar elements.

### BRIEF DESCRIPTION OF THE DRAWINGS

FIG. 1A shows a magnetocaloric substance without an applied magnetic field.

FIG. 1B shows a magnetocaloric substance with an applied magnetic field.

FIG. 2A shows a magnetocaloric vector and a magnetocaloric substance.

FIG. 2B shows a magnetocaloric substance bound to a receptor on the magnetocaloric vector's surface.

FIG. 2C shows a magnetocaloric vector that has begun to envelop a magnetocaloric substance bound to a receptor on the magnetocaloric vector's surface.

FIG. 2D shows a magnetocaloric vector that has formed a vacuole around a magnetocaloric substance.

FIG. 2E shows a magnetocaloric vector containing a magnetocaloric substance.

FIG. 3 a syringe for administering the magnetocaloric vector.

FIG. 4 shows a top view of a skull with the skin retracted and an opening in the skull.

FIG. 5 shows a top view of the skull with a syringe inserted through a cranial opening to a target location in the cranial vault.

FIG. 6 shows a perspective view of a syringe inserted through a cranial opening to a target location in the cranial vault.

FIG. 7A shows magnetocaloric vectors before migration to a cancerous cell.

FIG. 7B shows magnetocaloric vectors after migration to a cancerous cell.

FIG. 7C shows magnetocaloric vectors with an applied magnetic field.

FIG. 8 shows a device generating a magnetic field around a patient.

FIG. 9 shows a vector device for preparing a magnetocaloric vector according to an embodiment.

FIG. 10 shows a flowchart depicting a method for treating a patient with magnetocaloric vectors.

FIG. 11 shows a flowchart depicting a method for treating a patient with magnetocaloric vectors.

### DETAILED DESCRIPTION OF THE INVENTION

Infiltrative brain tumors, such as glioblastoma multiforme, are resistant to conventional cancer interventions and fewer than 5% of glioblastoma patients survive more than five years after diagnosis. Cancerous tumors are traditionally treated by surgically resecting the cancerous tissue followed by chemotherapy or radiation therapy to prevent the tumor from returning. However, treating a brain tumor is complicated by a need to balance removing the tumor against preserving brain tissue that is necessary for the patient's cognitive, motor, sensory, visual and other functions. Infiltrative brain tumors grow rapidly and the tumor can reconstitute itself if small amounts of viable tumor cells are left in the patient. Given the challenges in surgically removing an infiltrative brain tumor, up to 10% of the tumor can be left in the patient after a typical surgery. Even after all visualized tumor tissue has been removed, microscopic infiltrative tumor cells can be present throughout the patient's brain.

Unfortunately, most forms of chemotherapy can be ineffective against infiltrative brain tumors because the blood brain barrier can prevent chemotherapy from reaching the tumor cells in the central nervous system. Additionally, treatments that can pass through the blood brain barrier are often ineffective. For instance, temozolomide, the standard chemotherapy treatment for glioblastomas, can cross the blood brain barrier, but tumor cells can produce a protein (e.g., O6-methylguanine-DNA methyltransferase (MGMT)) that repairs DNA and mitigates the chemical's effectiveness. Bypassing the blood brain barrier, by infusing chemotherapy into the cerebrospinal fluid (CSF), is problematic because many of the chemicals involved in chemotherapy cannot easily diffuse from the CSF to the tumor cells. Radiation therapy is also ineffective because infiltrative brain tumors can efficiently repair radiation damage. Both chemotherapy and radiation therapy are most effective against dividing tumor cells. Some infiltrating tumor cells, particularly tumor stem cells, can exist in a senescent non-dividing state which is refractory to these treatments. Accordingly, a treatment that can target and kill infiltrative brain tumor cells is desirable.

The proposed treatment for infiltrative brain tumors includes using vectors carrying magnetocaloric substances to heat and kill tumor cells. A vector can be a chemical, polymer, cell, organism, a virus, antibody, etc. The magnetocaloric effect is when a substance (e.g., magnetocaloric substance) experiences a change in temperature due to a magnetic field. The substance's magnetic dipoles are initially oriented randomly, but the dipoles will align themselves along an applied magnetic field. As a result, the magnetocaloric substance's magnetic entropy and heat capacity are reduced, but, because the total energy remains unchanged, the substance radiates heat.

After surgically removing a tumor, a magnetocaloric vector can be infused into the target area (e.g., the cavity left after tumor resection). This vector can be selected for its tendency to migrate towards and to tumor cells. After infusion, the magnetocaloric vector migrates to the tumor cells. Various vectors can be used to deliver the magnetocaloric substance to tumor cells. For instance, mesenchymal stem cells have been shown to migrate to tumor cells in vivo. The vector delivers the magnetocaloric substance to the tumor cells and an applied magnetic field can be used to heat the substance. The magnetic field pulses can be controlled to maintain a target temperature for a target duration that is sufficient to kill the cancer cells.

The magnetic field can be controlled to maintain a target temperature for a target duration that is sufficient to stress the cancer cells sufficiently so that the cells can then be more readily killed using synergistic therapeutics. For instance, heating cancer cells may cause some of the cell's proteins to denature, driving increased proteotoxic stress. This could make the cells more dependent upon the proteasome—a major means of degrading and recycling defective proteins—for survival. This would render proteasome inhibitors, such as Bortezomib, an existing cancer therapy, more effective in the inhibitor's ability to kill cancer cells. The magnetocaloric vector may include autophagacy inhibitors.

In some implementations, a patient undergoes surgery to resect a tumor. The surgeon removes as much of the tumor as is possible given the circumstances and, after surgery, the doctor sends a sample to pathology for analysis. A standard histopathologic analysis and genetic screening can indicate that the tumor is a glioblastoma or other infiltrative brain tumor. Based on the diagnosis, the surgeon begins to prepare a magnetocaloric vector, in this case mesenchymal stem cells, and removes a fat sample from the patient. The sample is spun in a centrifuge to isolate the stem cells. After isolation, the stem cells are induced to phagocytize or otherwise absorb a magnetocaloric substance (e.g., gadolinium nanoparticles) to produce a magnetocaloric vector.

Approximately a week post-surgery, the patient undergoes a separate treatment to administer a magnetocaloric therapy. A surgeon creates an incision in the patient's head at an insertion point and retracts the scalp to expose the skull. The surgeon creates a burr hole (e.g., an opening in the skull) at the insertion point and cuts through the dura mater and arachnoid (e.g., tissue layers surrounding the brain). Using frame based or frameless stereotactic techniques, the surgeon inserts a stereotactic needle attached to a syringe through the burr hole to the target location and injects the magnetocaloric vector. After injecting the vector, the needle is withdrawn and the scalp is closed and the magnetocaloric vector is allowed to migrate to the cancerous cells for a period of time, such as, for example, a week. Once the vector has migrated to the cancerous cells, a pulsed magnetic field is generated around the patient. The magnetic field heats the magnetocaloric vector, thereby killing or substantively damaging the tumorous cells.

### I. The Magnetocaloric Substance

The magnetocaloric substance can be a metal that increases in temperature when exposed to a magnetic field. The permanent magnetic properties of a substance can be lost at a critical temperature (e.g., curie point). Above this point, the magnetic poles are aligned randomly but magnetism can be induced with an applied magnetic field. When the substance's poles are aligned by a magnetic field, the substance's entropy and heat capacity are reduced and as a result, the substance's temperature increases. In some embodiments, the magnetocaloric substance can be a class 2 magnetocaloric metal, and in some embodiments, the magnetocaloric metal can have a curie point from 27 C to 47 C. In some embodiments, the magnetocaloric substance can be nanoparticles of a magnetocaloric metal. It can also be a magnetocaloric substance of any kind which can be taken up by the vector.

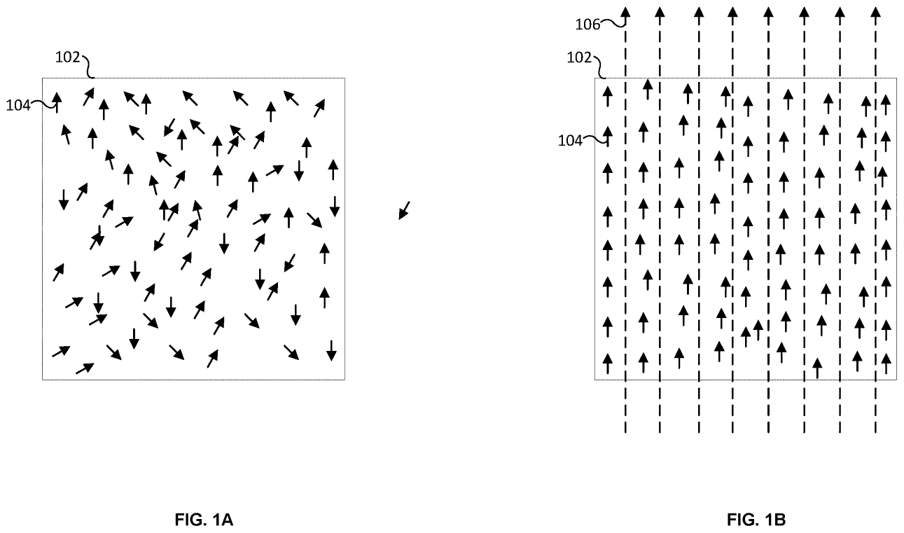

FIG. 1A shows a magnetocaloric substance 102 without an applied magnetic field. The magnetocaloric substance can be a gadolinium alloy, a praseodymium alloy, or any other substance that changes temperature when exposed to a magnetic field. For example, the magnetocaloric substance can be a gadolinium alloy, an iron alloy, a rare earth metal, Heusler alloy, Lanthanum alloy, etc. Gadolinium is a metal that is used as a contrast agent in medical imaging. Without an applied magnetic field, the magnetocaloric substance's magnetic dipoles 104 are randomly oriented.

FIG. 1B shows a magnetocaloric substance 102 with an applied magnetic field 106. The magnetocaloric substance's entropy and heat capacity are reduced as the magnetic field 106 orients the substance's dipoles 104. Because the magnetocaloric substance's energy remains constant, the change in entropy and specific heat result in the substance increasing in temperature.

### II. The Magnetocaloric Vector

The temperature change necessary to kill tumor cells can also be fatal to noncancerous tissue. In order to minimize the damage to healthy cells, the magnetocaloric substance can be delivered directly to the cancerous tissue using a magnetocaloric vector. The magnetocaloric vector can migrate through brain, or other tissue, to areas of microscopic infiltrating tumor and invade the tumor tissue or the vector can form a layer between the tumor cells and the parenchyma. Magnetocaloric vectors can include autologous stem cells (e.g., mesenchymal stem cells), induced pluripotent stem cells, neural stem cells, polymers, monoclonal antibodies, engineered proteins, exosomes, or naturally occurring proteins with affinity for tumor cells (e.g. ligands for surface receptors expressed on tumor cells). Autologous stem cells, such as mesenchymal stem cells, can be produced by isolating stem cells from a tissue sample obtained from the patient. The stem cells can be isolated from the tissue sample through centrifugation. Autologous stem cells can also be mobilized by pharmacologic means and collected from the blood (e.g. mobilization of hematopoietic stem cells into the blood and collection via apheresis). Stem cells can also be obtained in these same techniques from compatible donors and are referred to as allogenic stem cells. Induced pluripotent stem cells (iPSCs) can be produced from somatic cells, for instance, by transforming the somatic cells with Yamanaka factors (e.g, Oct3/4, Sox2, Klf4, and c-Myc). The iPSCs can be induced to differentiate into other stem cell types including neural stem cells.

Cellular vectors, such as stem cells, can be induced to phagocytose, pinocytose, passively uptake, or otherwise ingest or uptake magnetocaloric substances. Magnetocaloric substances could also be injected or otherwise driven into the stem cells. Stem cells have been shown in vivo to migrate to cancerous tissue. The cellular vector can uptake the magnetocaloric substances by incubating the vectors in a solution containing the magnetocaloric vectors. The vectors can be incubated for any of the following times: 1 minutes (min), 5 min, 10 min, 15 min, 20 min, 25 min, 30 min, 40 min, 50 min, 1 hour, 1.5 hours, 2 hours, 3 hours, 4 hours, 5 hours, 6 hours 7, hours, 8 hours, 9 hours, 10 hours, 11 hours, 12 hours, 18 hours, 1 day, 2 days, 3 days, 4 days, 5 days, 6 days, 1 week, etc. The concentration of magnetocaloric compound can be any of the following molar concentrations (mol/L): 1000, 100, 10, 1, 0.1, 0.01, 10⁻³, 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶, 10⁻¹⁷, 10⁻¹⁸, 10⁻¹⁹, 10⁻²⁰, 10⁻²¹, 10⁻²², 10⁻²³, 10⁻²⁴, 10⁻²⁵, 10⁻²⁶, etc. The temperature during incubation can be maintained at any of the following temperatures 0 degrees Celsius (° C.), 1° C., 2° C., 3° C., 4° C., 5° C., 6° C., 7° C., 8° C., 9° C., 10° C., 15° C., 20° C., 25° C., 30° C., 35° C., 40° C., 45° C., 50° C., 55° C., 60° C., 65° C., 70° C., 75° C., 80° C., 85° C., 90° C., 95° C., etc. Other techniques for transporting the magnetocaloric substance are contemplated and, for instance, the magnetocaloric substance could be transported on the exterior surface of the magnetocaloric vector. An advantage of using a magnetocaloric vector is that cancerous cells can be difficult to locate and a vector can migrate to the tumor even when the cell's location is unknown. An advantage of using a vector is that a magnetocaloric substance delivered directly to brain tissue or cerebrospinal fluid may not stay in the location where the substance was delivered.

The magnetocaloric vector can be induced to uptake medicines or other chemicals in addition to the magnetocaloric substance. For instance, the magnetocaloric vector can uptake one or more chemotherapy chemicals. An alternating magnetic field can be applied to the magnetocaloric vectors containing the magnetocaloric substance and one or more chemotherapy chemicals. The heat generated by the magnetocaloric substance and the alternating magnetic field can kill the magnetocaloric vectors and deliver the one or more chemotherapy chemicals to tumor or target area. Heat-activatable anti-cancer (e.g., chemotherapy) chemicals could also be developed, such that the heat from the magnetocaloric substance can induce these chemicals to from an inactive to an active state only in the area where a temperature increase is created.

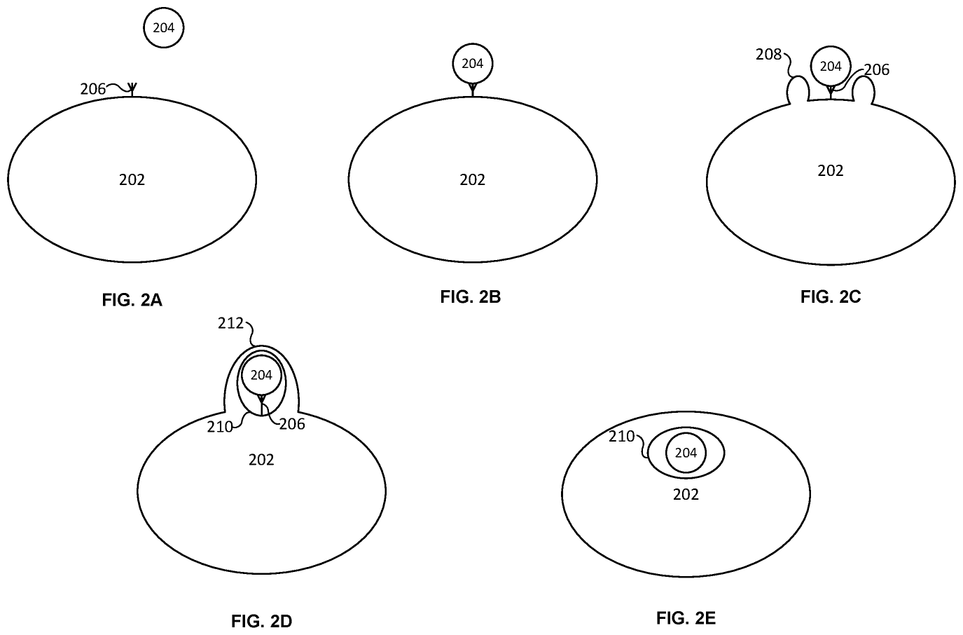

FIG. 2A shows a magnetocaloric vector 202, in this case a stem cell such as a mesenchymal stem cell, and a magnetocaloric substance 204. The magnetocaloric substance can be gadolinium, a gadolinium alloy, or an iron oxide. The magnetocaloric vector 202 can have a receptor 206 that can bind with the magnetocaloric substance.

FIG. 2B shows a magnetocaloric vector 202 with the magnetocaloric substance 204 bound to the receptor 206. However, the phagocytosis could be receptor mediated phagocytosis or non-receptor mediated phagocytosis. It could also be pinocytosis or any other form of endocytosis or cellular uptake. The magnetocaloric vector may transport the magnetocaloric substance without phagocytizing the substance. For instance, the magnetocaloric substance can be transported while the substance is bound to one or more receptors on the surface of the magnetocaloric vector.

FIG. 2C shows a magnetocaloric vector 202 as the cellular membrane 208 begins to envelop the magnetocaloric substance 204 attached to the receptor 206. In some circumstances, the magnetocaloric substance can enter magnetocaloric vector 202 via a pore created in the cell membrane in a process similar to the those used to induce the uptake of deoxyribonucleic acid (DNA) in techniques for inducing transformation (e.g., via the heat shock technique or electroporation). The magnetocaloric substance may enter magnetocaloric vector 202 via a membrane transport protein such as a carrier or channel. The magnetocaloric substance may be a nanoparticle or other substance that can pass directly through the cell membrane without a channel, transport protein, pore, or other means of transporting through the cell membrane. The magnetocaloric substance could be driven into the cell via microinjection or related techniques, or the magnetocaloric substance could be surrounded by a membrane or suspension and induced to fuse with the vector.

FIG. 2D shows a magnetocaloric vector 202 with the magnetocaloric substance 204 and receptor 206 enclosed in a vacuole 210. The vacuole 210 is contained in a protrusion 212 formed when the cellular membrane wrapped around the magnetocaloric substance 204. The magnetocaloric substance can be stored in the cytosol or in an organelle.

FIG. 2E shows the magnetocaloric vector 202 with the magnetocaloric substance 204 encased in a vacuole 210. The magnetocaloric substance may be stored in the cytosol or in cell organelles.

### III. Treatment

A treatment to kill infiltrative brain cells using a magnetocaloric vector can include surgically delivering the magnetocaloric vector to a target location inside the central nervous system. Delivering the magnetocaloric vector to the target location reduces the chance that the blood brain barrier can prevent the vector from reaching the cancerous cells. However, in some implementations the vector can be delivered intravenously or to the cerebrospinal fluid via nonsurgical means. The migration of intravenously delivered magnetocaloric vectors across the blood brain barrier can be enhanced by focal disruption of the blood brain barrier by techniques including focused ultrasound. The target location can be the cavity left by tumor removal. After waiting for the vector to migrate to the cancerous cells, a magnetic field can be applied to heat and kill or stress the tumor cells or deliver or activate synergistic chemicals. Brain tumors can be genetically heterogeneous and different cancerous cells can have separate genotypes within a single patient. The tumor's heterogeneity can complicate chemotherapy's efficacy because tumors with different genotypes may be susceptible to or resistant to different chemotherapeutic interventions. In contrast, heat is an effective treatment across tumor genotypes and is used surgically during tumor removal (e.g., laser ablation).

The process of preparing and administering a magnetocaloric vector can occur entirely within a surgical suite or operating room. Without leaving the operating room: a tissue sample, including blood, fat, bone marrow, or CSF, can be obtained from a patient; a magnetocaloric vector can be isolated from the tissue sample; the vector can be induced to transport the magnetocaloric substance (e.g., by phagocytosis); and the magnetocaloric vector can be administered to the patient (e.g., delivered to the target site). Performing these steps within the operating suite can reduce the risk that there is cross contamination between patients. These techniques can be performed by a device that is configured to process a patient tissue sample to extract stem cells and induce the stem cells to ingest the magnetocaloric substance.

The magnetocaloric vector can be prepared at a facility and delivered to a treatment site, such as a hospital or clinic, where the magnetocaloric vector can be administered to a patient. For instance, if the magnetocaloric vector is a stem cell, the cells can be isolated from tissue, cultured from an existing population of stem cells, or produced from other cell types (e.g., preparing iPSCs from skin cells). The magnetocaloric vector can be induced to uptake the magnetocaloric substance as described herein. The magnetocaloric vector, with the magnetocaloric substance, can be prepared for transportation using cryopreservation techniques such as those disclosed in Jang T H, Park S C, Yang J H, Kim J Y, Seok J H, Park U S, Choi C W, Lee S R, Han J. Cryopreservation and its clinical applications. Integr Med Res. 2017 March; 6(1):12-18. doi: 10.1016/j.imr.2016.12.001. Epub 2017 Jan. 10. PMID: 28462139; PMCID: PMC5395684; and Uhrig M, Ezquer F, Ezquer M. Improving Cell Recovery: Freezing and Thawing Optimization of Induced Pluripotent Stem Cells. Cells. 2022 Feb. 24; 11(5):799. doi: 10.3390/cells11050799. PMID: 35269421; PMCID: PMC8909336.

After thawing, the magnetocaloric vector may be allowed to rest before the vector is administered to a patient. The magnetocaloric vector may be rested at a suitable temperature such as any of the following temperatures 0 degrees Celsius (° C.), 1° C., 2° C., 3° C., 4° C., 5° C., 6° C., 7° C., 8° C., 9° C., 10° C., 15° C., 20° C., 25° C., 30° C., 35° C., 40° C., 45° C., 50° C., 55° C., 60° C., 65° C., 70° C., 75° C., 80° C., 85° C., 90° C., 95° C., etc. The temperature may vary through the resting process. Before administration, the magnetocaloric vector may rest for 30 minutes, 1 hour, 2 hours, 3 hours, 4 hours, 5 hours, 6, hours, 7 hours, 8 hours, 9 hours, 10 hours, 11 hours, 12 hours, 14 hours, 16 hours, 18 hours, 20 hours, 22 hours, 24 hours, 1.5 days, 2 days, 3 days, 4 days, 5 days, 6 days, 1 week, 2 weeks, etc. The magnetocaloric vector may be transported as in a liquid media as described in Yu, Na-Hee, et al. “Optimal stem cell transporting conditions to maintain cell viability and characteristics.” Tissue Engineering and Regenerative Medicine 15.5 (2018): 639-647. The magnetocaloric vector may be transported while encapsulated in a hydrogel as described in Wright, Bernice, et al. “Enhanced viability of corneal epithelial cells for efficient transport/storage using a structurally modified calcium alginate hydrogel.” Regenerative medicine 7.3 (2012): 295-307.

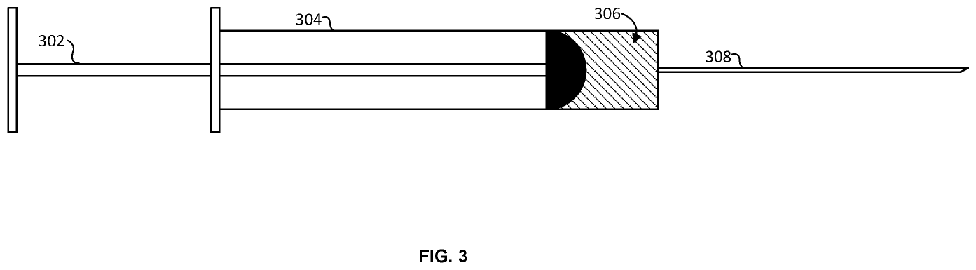

FIG. 3 shows a stereotactic biopsy needle attached to a syringe 300 for delivering the magnetocaloric vector to a target location. The syringe can be configured for frameless stereotactic surgery or stereotactic surgery using a frame. Stereotactic surgery involves using a coordinate system to accurately deliver a needle to a specific location in the body. Stereotactic techniques can be used to inject a substance or remove a biopsy. The needle includes a plunger 302, a barrel 304 configured to couple with the plunger, a reservoir 306 formed by the plunger 302 and barrel 304, and a needle 308. The needle can be configured to allow a tissue biopsy at the target site. The needle 308 can be inserted to the target location and the magnetocaloric vector, stored in the reservoir 306, can be injected by depressing the plunger 302.

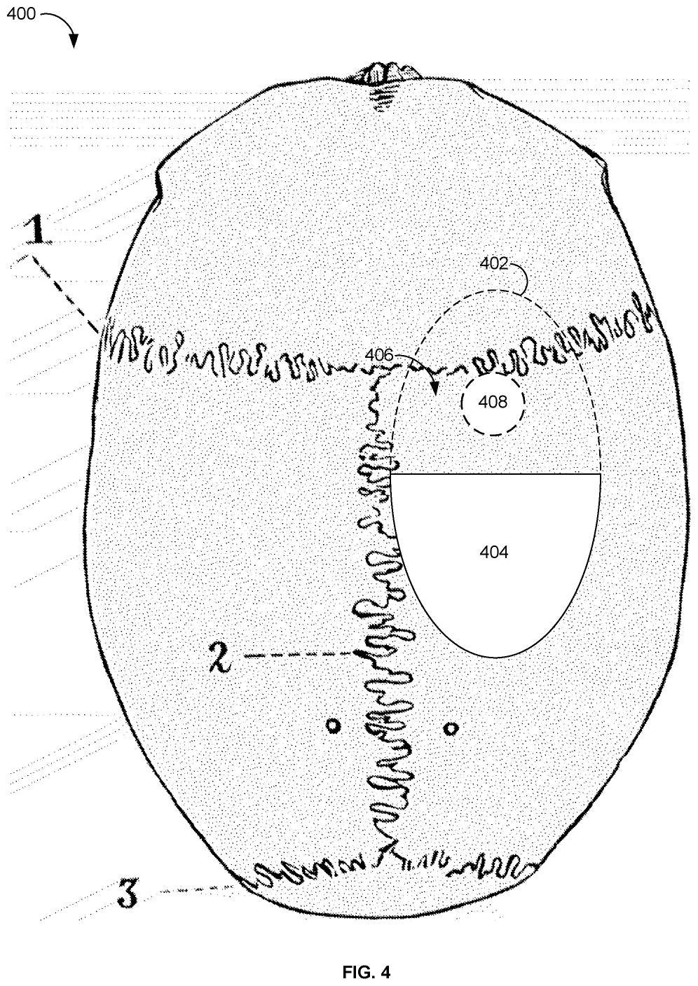

FIG. 4 shows an axial view of a skull 400 with an incision and a cranial opening. An incision 402 can be made in the scalp and a section of scalp 404 can be retracted to expose the skull 406. An opening 408 can be made in the skull so that a stereotactic needle can be inserted to the target location in the cranial vault. In some implementations, the magnetocaloric vector can be delivered using a catheter inserted to the target location. In other implementations, a reservoir (e.g., Ommaya reservoir or other reservoir) can be coupled with an inserted catheter and implanted into the cranial opening 408 to facilitate repeated magnetocaloric vector infusions. In some embodiments, a reservoir such as the device disclosed in U.S. Application No. 63/226,624 (Attorney Docket No.: 107879-1249451-000100US), entitled “Reservoir for Continuous Infusion of Material into Cerebrospinal Fluid Spaces”, and filed on Jul. 29, 2021 the entirety of which is hereby incorporate by reference herein.

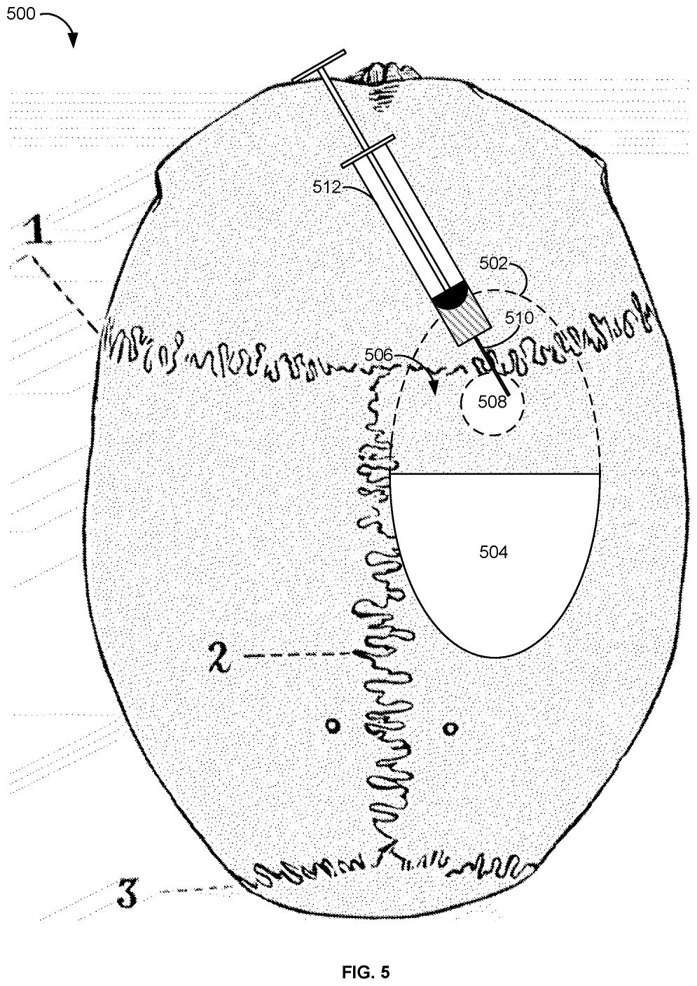

FIG. 5 shows an axial view of the skull 500 with a syringe inserted into a cranial opening. An incision in the scalp 502 can allow a section of scalp 504 to be retracted to expose the skull 506. A cranial opening 508 can be made in the skull 506 to allow a stereotactic needle 510, attached to a syringe 512, to be inserted to a target location in the cranial vault.

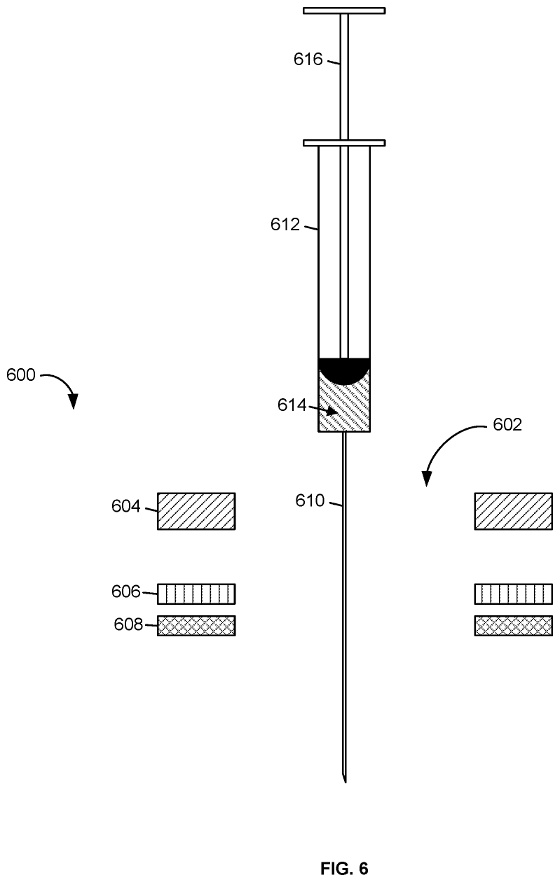

FIG. 6 shows a perspective view of a stereotactic needle 600 inserted through a cranial opening to a target location. An opening 602 can be made by drilling through the skull 604, opening the dura mater 606, and opening the arachnoid 608. A stereotactic needle 610 can be inserted through the opening 602 to the target location. The stereotactic needle 610 can be attached to a syringe 612 and by depressing the plunger 614, the magnetocaloric vector in the syringe's reservoir can be delivered to the target location. The magnetocaloric vector can be suspended in a liquid (e.g., a saline solution) during injection. Continuous injection over an extended time period can be achieved by connecting the syringe to an infusion pump.

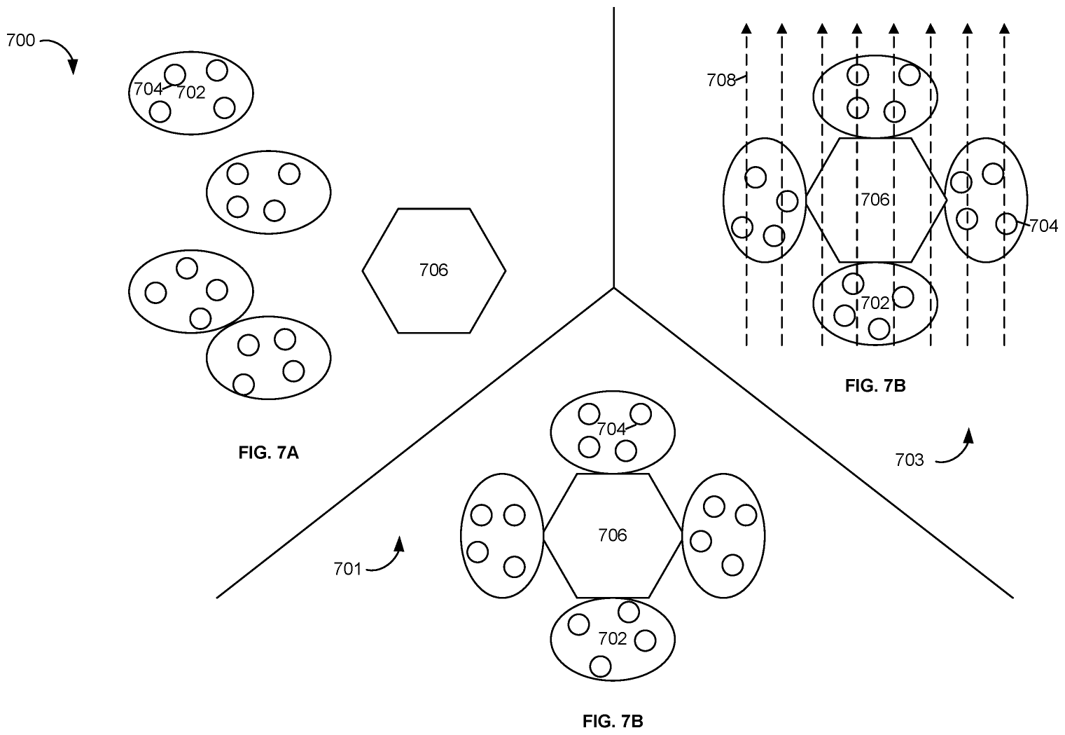

FIG. 7A shows the magnetocaloric vectors before the vectors migrate to the cancerous cells. The vectors 702 can contain the magnetocaloric substance 704. The vectors can be an autologous stem cell, an induced pluripotent stem cell, neural stem cells, allogenic stem cells, immune cells, other cell types, monoclonal antibodies, polymers, synthetic proteins, viruses, virus-like particles, engineered viruses, engineered proteins, naturally occurring proteins with affinity for cancer cells, or exosomes. The cancerous cell 706 can be any infiltrative tumor, infiltrative brain tumors (e.g., glioma, astrocytoma, oligodendroglioma, ependymoma, meningioma, medulloblastoma, ganglioglioma, craniopharyngiomas, etc.), or other infiltrative tumors or cancers.

FIG. 7B shows a cancerous cell surrounded by magnetocaloric vectors containing a magnetocaloric substance. After being introduced to the target area, the magnetocaloric vectors 702, containing the magnetocaloric substance 704, migrate to the cancerous cells 706. The migration can take several days and migration generally occurs 4 to 14 days after the magnetocaloric vectors 702 are injected into the target area. Magnetocaloric vectors 702 can infiltrate the tumor and comingle with the tumor cells, or the vectors may form a layer between the tumor cells and healthy cells. Tissue containing magnetocaloric vectors 702 can be called a vector cluster.

Whether magnetocaloric vectors 702 are located within the tumor or whether the vectors are surrounding the tumor can depend on the length of time that has elapsed after the vectors were introduced to the target area. In some circumstances, magnetocaloric vector 702 can be introduced to the target area in multiple injections. For instance, a first injection can be made at day 0, a second injection can be made at day 3, and a magnetic field can be applied at day 7. Staggering injections can help to evenly distribute the magnetocaloric vector throughout the tumor. The injections can be separated by 1 hour, 6 hours, 8 hours, 12 hours, 18 hours. 1 day, 2 days, 3 days, 4 days, 6 days, 1 week, 1.5 weeks, 2 weeks, etc. In some circumstances, more than one type of magnetocaloric vector 702 can be used in one injection or multiple types of vectors can be used in a single injection.

FIG. 7C shows a magnetic field heating magnetocaloric vectors surrounding a cancerous cell. The magnetic field 708 can be a pulsed or continuous magnetic field. In some implementations, the magnetic field's pulsing can be controlled to maintain a target temperature in the areas surrounding the magnetocaloric vectors 702 containing the magnetocaloric substance 704. Heating the magnetocaloric substance 704 can generate a target temperature for a target duration that is sufficient to kill the cancerous cell 706. In some implementations, cancerous cells 706 can be killed by a 46 degree Celsius (C) target temperature maintained for a ten minute target duration. The target temperature can be 40° C., 41° C., 42° C., 43° C., 44° C., 45° C., 46° C., 47° C., 48° C., 49° C., 50° C., 51° C., 52° C., 53° C., 54° C., 55° C., 55° C., etc.

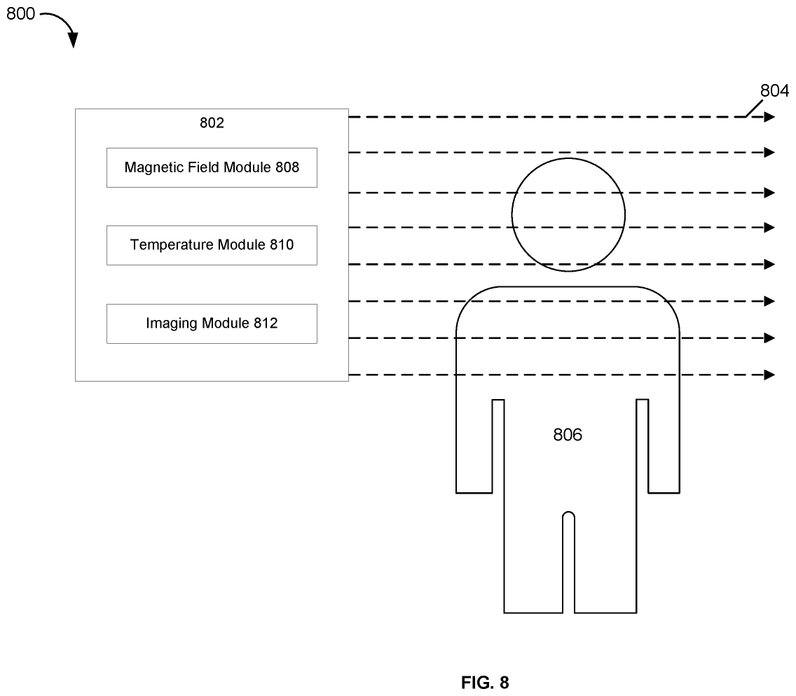

FIG. 8 shows an electromagnetic field generator creating a magnetic field around a patient. The magnetic field generator 802 can be configured to produce a magnetic field 804 around a patient 806. The magnetic field 804 can be a pulsed magnetic field, and, in some implementations, the pulsed magnetic field can be controlled with a magnetic field module 808 to maintain a target temperature around the magnetocaloric vectors. Multiple frequencies for the pulsed magnetic field are contemplated including 1 Hertz (Hz), 10 Hz, 50 Hz, 100 Hz, 250 Hz, 500 Hz, 1000 Hz, 10,000 Hz, 100,000 Hz, 1,000,000 Hz, 10,000,000 Hz, 100,000,000 Hz, 500,000,000 Hz, 1,000,000,000 Hz, etc. Magnetic field generator 802 may generate a magnetic field while being in physical contact with patient 806 (e.g., touching the patient). For instance, magnetic field generator 802 may touch the head of patient 806 so that magnetic field 804 passes directly from the generator to the patient.

The magnetocaloric vectors may be used to deliver chemical compounds to the tumor site. For example, a magnetocaloric vector may contain chemotherapy chemicals in addition to the magnetocaloric vectors. An applied magnetic field may be used to rupture the magnetocaloric vector and deliver the chemotherapy chemicals to the tumor. The applied magnetic field can be timed so that the chemotherapy chemical is delivered at an optimal time (e.g., when the tumor cells have infiltrated the tumor or a desired amount of time after tumor resection). The applied magnetic field can kill cells used to deliver chemotherapy chemicals, or other substances, to mitigate the risk that the cells cause unintended effects after delivering the chemotherapy chemicals (e.g., concerns that the cells form tumors).

The magnetic field generator can be a magnetic resonance imaging (MM) device or a Mill device that has been altered to produce a desired pulsed magnetic field. The temperature around the magnetocaloric vectors can be measured using a temperature module 810 employing known methods for magnetic resonance thermometry (e.g., proton resonance frequency). In some implementations, the magnetocaloric substance is visible on an Mill scan and the magnetocaloric vector's location can be verified with the imaging module 812 through Mill imaging. In such an embodiment, imaging of the magnetocaloric vector can indicate the location of cancer cells as the magnetocaloric vector can migrate to those cells.

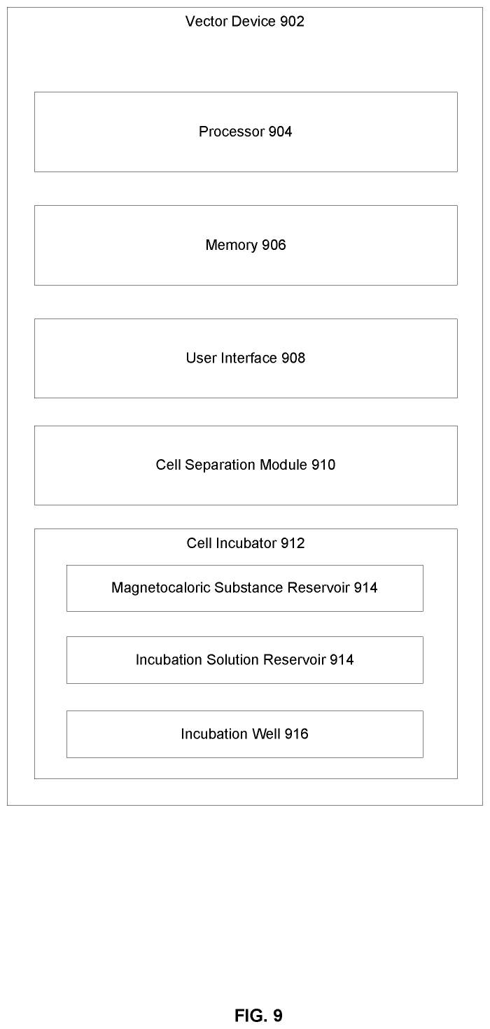

FIG. 9 shows a vector device for preparing a magnetocaloric vector according to an embodiment. The vector device 902 can be used to process a patient tissue sample to prepare a magnetocaloric vector. Vector device 902 can be located in an operating room and the tissue sample can be processed without leaving the operating room. Processor 904 can be one or more processors that are configured to perform instructions stored in memory 906. The instructions can comprise one or more of the techniques described herein. Input to vector device 902 can be provided via user interface 908. The memory can be non-volatile memory such as a solid state drive, a hard disk drive, cache memory, random access memory, etc.

A tissue sample, such as a bone marrow or fat sample, can be provided to the cell separation module 910. Cell separation module 910 can be a centrifuge or an automated cell separator, that uses electric charge or magnetic field to isolate cell types, to separate magnetocaloric vectors, such as stem cells or another suitable cell type, from the tissue sample. The isolated magnetocaloric vector can be added to the incubation well 916 in the cell incubator 912. Incubation well 916 can be an enclosure that can be used to induce the magnetocaloric vector to absorb, uptake, or otherwise ingest the magnetocaloric substance. Incubation well 916 can contain a heating element or cooling element to control the temperature during incubation. Cell incubator 912 can include a magnetocaloric substance reservoir 914 containing a magnetocaloric substance and an incubation solution reservoir containing a solution for incubating the magnetocaloric vector. Magnetocaloric substance reservoir 914 and incubation solution reservoir 914 can provide the magnetocaloric substance and solution to the incubation well 916 containing the magnetocaloric vector. The magnetocaloric substance and solution can be provided in concentrations that are suitable to induce the magnetocaloric vector to intake the magnetic substance during incubation. After incubation, the solution containing the magnetocaloric vector can be removed from incubation well 916 and introduced to target area as described herein.

### IV. Method Flow

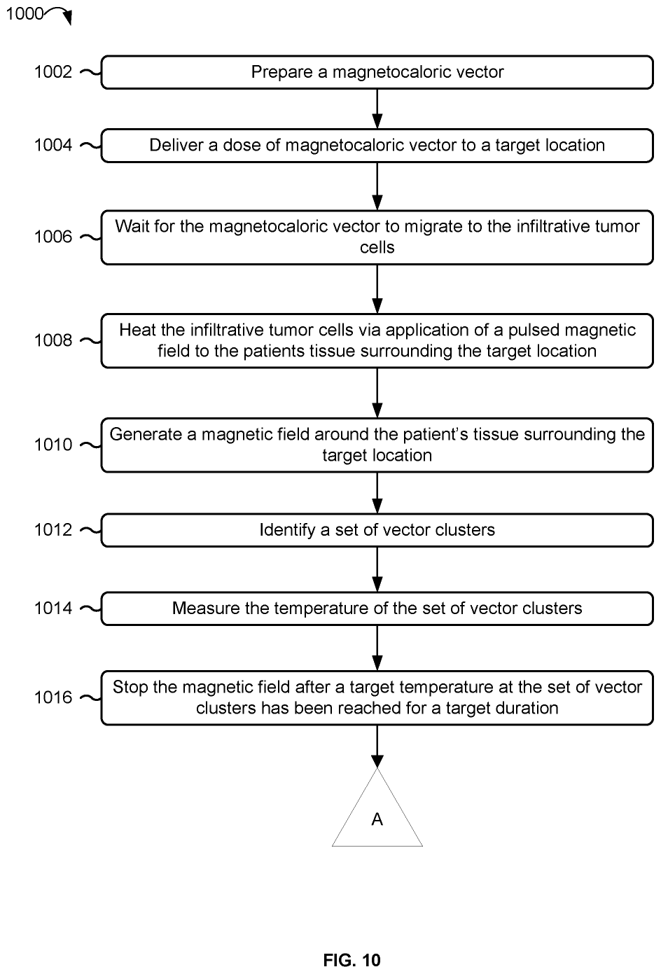

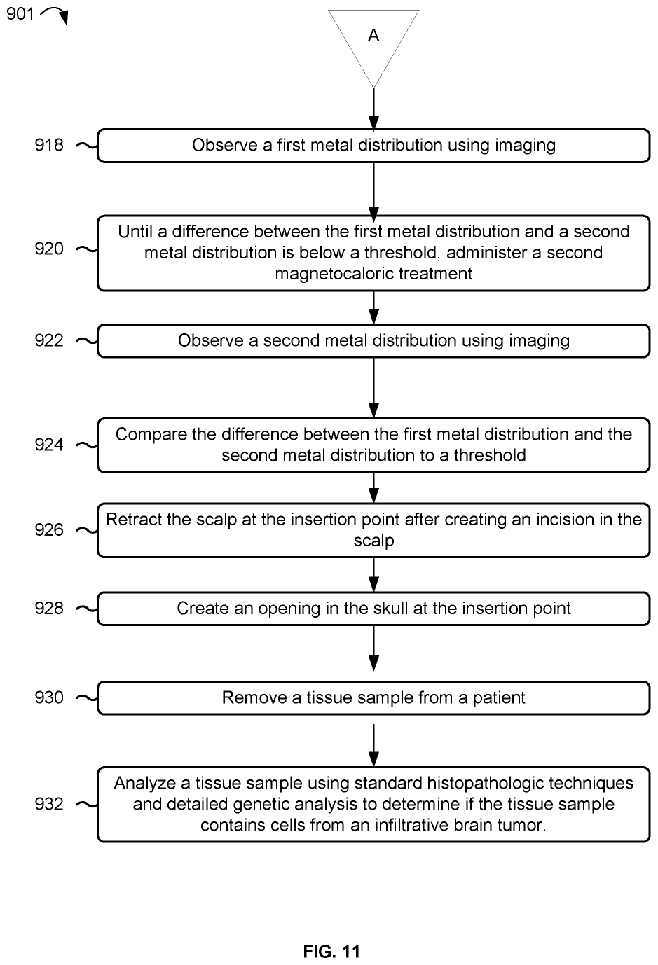

FIG. 10 illustrates a simplified flow diagram of a method for treating infiltrative brain tumors or other infiltrative tumors with magnetocaloric vectors that spans FIGS. 10 and 11. Process 1000 of FIG. 10 may correspond to a first phase of the process, while process 1001 of FIG. 11 may correspond to a second phase of the process.

At block 1002, process 1000 may include preparing a magnetocaloric vector. The magnetocaloric vector can be a monoclonal antibody, a polymer, an autologous stem cell, an induced pluripotent stem cell, a neural stem cell or an exosome (e.g., lipid bilayer). The vectors can be an autologous stem cell, an induced pluripotent stem cell, neural stem cells, allogenic stem cells, immune cells, other cell types, monoclonal antibodies, polymers, synthetic proteins, viruses, virus-like particles, engineered viruses, engineered proteins, naturally occurring proteins with affinity for cancer cells, or exosomes. The magnetocaloric vector can contain a magnetocaloric substance. An autologous stem cell can be a mesenchymal stem cell. The magnetocaloric substance can be a magnetocaloric metal, and can be a class 1 or class 2 magnetocaloric metal. In some circumstances, a mixture of two or more magnetocaloric metals can be used. The magnetocaloric metal can have a curie point from 27 C to 47 C.

At block 1004, process 1000 may include delivering a dose of magnetocaloric vector to a target location. The target location can be a cavity left by tumor removal, a ventricle, or an infusion into the brain tissue. The magnetocaloric vector can be used as a primary treatment without the tumor being resected before the magnetocaloric vector is administered to the target area. For instance, the vector could be administered into the brain tissue, CSF, blood, or other tissue without resecting a tumor first. The magnetocaloric vector may be administered and the tumor can be subsequently resected. For example, the magnetocaloric vector can be administered before the tumor resection so that the magnetic field could be applied on the same day as the resection surgery (e.g., because the vector migrated to the tumor prior to the resection surgery). The dose can be delivered to the target location using stereotactic surgical techniques, and/or via a reservoir. The stereotactic surgical techniques can utilize a frame (e.g., simple orthogonal system, burr hole mounted system, arc-quadrant system, arc-phantom system, etc.). The magnetocaloric vector may be delivered intravenously. In some circumstances, the magnetocaloric vector may be administered in staggered doses that are hours or days apart. Other methods of administering the magnetocaloric vector are contemplated such as via intravenous injection, an interstitial injection, an injection into a body cavity, an injection into the CSF.

At block 1006, process 1000 may include waiting for the magnetocaloric vector to migrate to the infiltrative tumor cells. The waiting time can be between 1 and 28 days, between 2 and 21 days, and/or between 4 and 14 days. The tumor cells can include infiltrative tumors such as (glioblastomas liver cancers, metastases, etc.), tumor cells, and cells in precancerous lesions (e.g. carcinoma in situ).

At block 1008, process 1000 may include heating infiltrative tumor cells via application of a magnetic field to tissue of the patient surrounding the target location. In some implementations, the magnetic field may be pulsed or continuous. For example, the magnetic field could be an alternating magnetic field. The magnetic field pulses' frequency, duty cycle, or amplitude can be varied to control the energy and/or temperature delivered by the magnetocaloric substance. The magnetic field can be delivered by a device that is in close proximity with the patient's body or physically in contact with the patient's body (e.g., touching the scalp, skull, brain tissue, etc.).

Although FIG. 10 shows example blocks of process 1000, in some implementations, process 1000 may include additional blocks, fewer blocks, different blocks, or differently arranged blocks than those depicted in FIG. 10. Additionally, or alternatively, two or more of the blocks of process 1000 may be performed in parallel.

At block 1010, alone or in combination with the first implementation, process 1000 can include generating a magnetic field around tissue of the patient surrounding the target location. In some implementations, the magnetic field can be generated around the patient's central nervous system. The magnetic field can be generated by, for example, an Mill device.

At block 1012, process 1000 can include identifying a set of vector clusters. A vector cluster can be tissue containing magnetocaloric vectors. The vector clusters can be formed by magnetocaloric vectors forming a layer between tumor cells and the parenchyma. The vector clusters can also be formed by the magnetocaloric vectors infiltrating a tumor and/or migrating to the tumor.

At block 1014, process 1000 can include measuring the temperature at the set of vector clusters. The temperature can be measured using magnetic resonance thermometry (e.g., photon resonance frequency).

At block 1016, process 1000 can include stopping the magnetic field after a target temperature at the set of vector clusters has been reached for a target duration. The target temperature can be 45-65 degrees C. for a target time of, for example, 0-45 minutes, 0-30 minutes, and/or 0-15 minutes.

FIG. 11 illustrates the second phase of the process in further detail. At block 1018, alone or in combination with one or more of the first or second implementations, process 1001 can further include observing a first metal distribution using imaging. In some implementations, the magnetocaloric substance is visible using standard imaging techniques (e.g., magnetic resonance imaging, x-ray imaging, computerized tomography, magnetoencephalography, etc.). In some implementations, a metal can be added to the magnetocaloric vector so that the vector is visible using standard imaging techniques.

At block 1020, process 1000 can include administering a second magnetocaloric treatment until a difference between the first metal distribution and a second metal distribution is below a threshold. In some embodiments, this second magnetocaloric treatment can be delivered to a second target location, which second target location can be the same target location as that of the first magnetocaloric treatment, and in some embodiments, the second target location can be a different target location than that of the first magnetocaloric treatment.

At block 1022, process 1000 can include observing the second metal distribution using imaging. The second metal distribution can be compared to the first metal distribution. A difference between the first metal distribution and the second metal distribution can be determined, which difference can be quantified in, for example, one or several difference scores. The difference between the first and second metal distributions, or more specifically, the one or several difference scores can be compared to a threshold and/or to a threshold value. When the difference score is above a threshold, the comparison between the difference score and threshold can indicate that cancerous cells were killed during the last magnetocaloric treatment and an additional round of treatment may be beneficial. When the difference score is below a threshold, the comparison may indicate that the last magnetocaloric treatment did not kill tumor cells and an additional round of magnetocaloric treatment can be unwarranted.

At block 1024, in conjunction with one or more of the first through third implementations, process 1000 can further include identifying an insertion point.

At block 1026, process 1000 can include retracting the scalp at the insertion point after creating an incision in the scalp.

At block 1028, process 1000 can include creating an opening in the skull at the insertion point. The insertion point can be a location on the skull that allows for the shortest path from the insertion point to the target location that avoids vasculature and eloquent cortex. The insertion point can be a historical safe insertion point. The insertion point can be identified with standard imaging techniques.

At block 1030, in conjunction with any of the first through fourth implementations, process 1000 can include removing a tissue sample from a patient.

At block 1032, process 1000 can include analyzing the tissue sample using standard histopathologic techniques and detailed genetic analysis to determine if the tissue sample contains cells from an infiltrative brain tumor.

The specific details of particular embodiments may be combined in any suitable manner or varied from those shown and described herein without departing from the spirit and scope of embodiments of the invention.

The above description of exemplary embodiments of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications to thereby enable others skilled in the art to best utilize the invention in various embodiments and with various modifications as are suited to the particular use contemplated.

All publications, patents, and patent applications cited herein are hereby incorporated by reference in their entirety for all purposes. Aspects of embodiments can be implemented in the form of control logic using hardware circuitry (e.g. an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

All patents, patent applications, publications, and descriptions mentioned herein are incorporated by reference in their entirety for all purposes. None is admitted to be prior art. Where a conflict exists between the instant application and a reference provided herein, the instant application shall dominate.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “‘1’ or ‘2’ or ‘3’ or ‘1 and 2’ or ‘1 and 3’ or ‘2 and 3’ or ‘1, 2 and 3’”.

Whenever a range is given in the specification, for example, a temperature range, a time range, or a composition range, all intermediate ranges and subranges, as well as all individual values included in the ranges given are intended to be included in the disclosure.

As used herein, “consisting essentially of” does not exclude materials or steps that do not materially affect the basic and novel characteristics of the claim.

What is claimed is:

1. A method for treating infiltrative tumor cells comprising:
administering a magnetocaloric vector to a patient in response to a determination of the presence of cells of an infiltrative tumor, wherein administering a magnetocaloric treatment comprises:
preparing a magnetocaloric vector;
delivering a dose of magnetocaloric vector to a target location;
waiting for the magnetocaloric vector to migrate to the infiltrative tumor cells; and
heating infiltrative tumor cells via application of a pulsed magnetic field to tissue of the patient surrounding the target location.

2. The method of claim 1, wherein applying a magnetic field to the tissue of the patient further comprises:
generating a magnetic field around the tissue of the patient surrounding the target location;
identifying a set of vector clusters;
measuring a temperature at the set of vector clusters; and
stopping the magnetic field after a target temperature at the set of vector clusters has been reached for a target duration.

3. The method of claim 1, wherein the magnetic field is controlled to maintain a target temperature at the target location.

4. The method of claim 1, wherein the magnetic field is controlled to heat a magnetocaloric substance for a target duration.

5. The method of claim 1, wherein the magnetocaloric vector is at least one of: an autologous stem cell, an induced pluripotent stem cell, a neural stem cell, an allogenic stem cell, an immune cell, a monoclonal antibody, a polymer, a synthetic protein, a virus, an engineered viruses, an engineered protein, a naturally occurring proteins with affinity for cancer cells, or an exosome.

6. The method of claim 1, wherein the magnetocaloric vector contains a magnetocaloric metal.

7. The method of claim 6, further comprising:
observing a first metal distribution using imaging;
until a difference between the first metal distribution and a second metal distribution is below a threshold:
administering a second magnetocaloric treatment;
observing a second metal distribution using imaging; and
comparing the difference between the first metal distribution and the second metal distribution to a threshold.

8. The method of claim 6, wherein the magnetocaloric vector contains a chemotherapy chemical.

9. The method of claim 6, wherein the magnetocaloric metal has a curie point of 27 C-47 C.

10. The method of claim 6, wherein preparing the magnetocaloric vector further comprises:
obtaining a tissue sample from the patient;
processing the tissue sample to isolate stem cells; and
inducing the stem cells to phagocytize the magnetocaloric metal.

11. The method of claim 10, wherein the method is performed within an operating room.

12. The method of claim 10, wherein the dose of the magnetocaloric vector is 5-25 million cells per kilogram of bodyweight.

13. The method of claim 1, wherein the method further includes:
identifying an insertion point;
retracting a scalp of the patient, after creating an incision, at the insertion point; and
creating an opening in a skull of the patient at the insertion point.

14. The method of claim 13, wherein the insertion point is a location on the skull that allows for the shortest path from the insertion point to the target location that avoids vasculature and eloquent cortex.

15. The method of claim 13, wherein the insertion point is a historical safe insertion point.

16. The method of claim 1, wherein the magnetocaloric vector contains a proteasome inhibitor.

17. The method of claim 1, wherein the method further includes:
removing a tissue sample from a patient; and
analyzing the tissue sample using standard histopathologic and detailed genetic analysis to determine if the tissue sample contains cells from an infiltrative tumor.

18. The method of claim 1, wherein the method includes waiting 4-14 days for the magnetocaloric vector to migrate to the infiltrative tumor cells.

19. The method of claim 1, wherein the magnetic field is generated by a magnetic field generator that is in physical contact with the patient.

20. A device comprising:
one or more processors configured to:
receive a tissue sample from a patient;
process the tissue sample to isolate stem cells; and
induce the stem cells to phagocytize a magnetocaloric metal.
